(19) United States
(12) Patent Application Publication
(10) Pub. No.: US 2023/0145882 A1
(43) Pub. Date: May 11, 2023

(54) THERMOELECTRIC MODULE AND THERMOELECTRIC DEVICE COMPRISING SAME

(71) Applicant: TEGWAY CO.,LTD, Daejeon (KR)

(72) Inventor: Ockkyun OH, Daejeon (KR)

(73) Assignee: TEGWAY CO.,LTD, Daejeon (KR)

(21) Appl. No.: 17/917,895

(22) PCT Filed: Jan. 21, 2021
(86) PCT No.: PCT/KR2021/000819

§ 371 Date: Oct. 7, 2022

(30) Foreign Application Priority Data
Apr. 9, 2020 (KR) 10-2020-0043444

(51) Int. Cl. H10N 10/17

(52) U.S. Cl. CPC H10N 10/17

(57) ABSTRACT

A thermoelectric device includes thermoelectric groups connected in series between first and second terminals provided in a plate shape deformable to a curved surface, having a first surface and an opposite second surface as a main surface, and configured to receive power from the outside. The thermoelectric groups include a first thermoelectric group configured to provide any one of a feeling of coldness and a feeling of warmth toward the first surface when the power is applied, an N-type semiconductor and a P-type semiconductor disposed between the first surface and the second surface, and an electrode configured to alternately connect an N-type semiconductor and a P-type semiconductor and disposed adjacent to any one surface of the main surface. The thermoelectric filters also include a second thermoelectric group providing the other of the feeling of coldness and the feeling of warmth toward the first surface when the power is applied.

### TECHNICAL FIELD

The present invention relates to a thermoelectric module and a thermoelectric device including the same, and specifically, to a thermoelectric module providing a thermal stimulus including a thermal grill illusion to a user and a thermoelectric device including the same.

### BACKGROUND ART

A thermoelectric element (TE) is an element which causes an exothermic reaction or an endothermic reaction by receiving electrical energy due to the Peltier effect, and is used to provide a thermal stimulus to a user.

A thermal stimulus that can be provided to a user by a thermoelectric module and a thermoelectric device including thermoelectric elements includes a feeling of warmth, a feeling of coldness, and a thermal grill illusion. Among the above, the thermal grill illusion means that the user perceives warm heat and cold heat as a sensation instead of individually perceiving the feeling of warmth and the feeling of coldness when the warm heat and the cold heat are simultaneously given.

Recently, research for providing the sensation to the user using such a thermal grill illusion in a VR/AR environment or other application methods have proceeded.

### DISCLOSURE

### Technical Problem

The present invention is directed to providing a thermoelectric module providing a thermal grill illusion and a thermoelectric device including the same.

The problems to be solved by the present invention are not limited to the above-described problems, and other problems which are not mentioned will be clearly understood by those skilled in the art from the present specification and the accompanying drawings.

### Technical Solution

One aspect of the present invention provides a thermoelectric device including a plurality of thermoelectric groups connected in series between a first terminal and a second terminal, the first terminal and the second terminal provided in a plate shape deformable to a curved surface, having a first surface and a second surface opposite the first surface as a main surface, and configured to receive power from the outside, including wherein the plurality of thermoelectric groups include, a first thermoelectric group configured to provide any one of a feeling of coldness and a feeling of warmth toward the first surface when the power is applied, and including an N-type semiconductor and a P-type semiconductor disposed between the first surface and the second surface, and an electrode configured to alternately connect an N-type semiconductor and a P-type semiconductor and disposed adjacent to any one surface of the main surface, and a second thermoelectric group providing the other of the feeling of coldness and the feeling of warmth toward the first surface when the power is applied, and including an N-type semiconductor and a P-type semiconductor disposed between the first surface and the second surface, and an electrode configured to alternately connect an N-type semiconductor and a P-type semiconductor and disposed adjacent to any one surface of the main surfaces, wherein one end electrically adjacent to the first terminal of an electrode disposed adjacent to the first surface among the electrode of the second thermoelectric group is connected to the P-type semiconductor, when one end electrically adjacent to the first terminal of an electrode disposed adjacent to the first surface among the electrode of the first thermoelectric group is connected to the N-type semiconductor, wherein the one end electrically adjacent to the first terminal of the electrode disposed adjacent to the first surface among the electrode of the second thermoelectric group is connected to the N-type semiconductor, when the one end electrically adjacent to the first terminal of the electrode disposed adjacent to the first surface among the electrode of the first thermoelectric group is connected to the P-type semiconductor, and wherein the first thermoelectric group and the second thermoelectric group are connected through a connection electrode, and the connection electrode connects the N-type semiconductor of the first thermoelectric group and the N-type semiconductor of the second thermoelectric group or connects the P-type semiconductor of the first thermoelectric group and the P-type semiconductor of the second thermoelectric group.

Solutions to the problems of the present invention are not limited to the above-described solutions, and solutions which are not mentioned will be clearly understood by those skilled in the art from the present specification and the accompanying drawings.

### Advantageous Effects

According to the present invention, a thermoelectric module providing a thermal grill illusion based on a connection relationship between thermoelectric elements and electrodes and a thermoelectric device including the same can be provided.

The effects of the present invention are not limited to the above-described effects, and effects which are not mentioned will be clearly understood by those skilled in the art from the present specification and the accompanying drawings.

### DESCRIPTION OF DRAWINGS

FIG. 1 is a view related to a configuration of a thermoelectric device according to one embodiment.

FIGS. 2 and 3 are views related to a thermoelectric module according to one embodiment.

FIG. 4 is a view related to thermoelectric elements according to one embodiment.

FIGS. 5 and 6 are views related to the connection between the thermoelectric elements, electrodes, and terminals according to one embodiment.

FIG. 7 is a view related to the thermoelectric module providing a thermal stimulus according to one embodiment.

FIG. 8 is a view related to an example of the thermoelectric module providing a thermal grill illusion according to one embodiment.

FIGS. 9 and 10 are views related to another example of the thermoelectric module providing the thermal grill illusion according to one embodiment.

FIG. 11 is a view related to still another example of the thermoelectric module providing the thermal grill illusion according to one embodiment.

FIGS. 12 and 13 are views related to a heat flow of a thermoelectric device providing a thermal grill illusion according to one embodiment.

FIG. 14 is a graph illustrating a temperature change of a heat dissipation module according to one embodiment.

FIG. 15 is a graph illustrating a temperature change of a heat dissipation module included in the thermoelectric device providing the thermal grill illusion according to one embodiment.

FIG. 16 is a view related to a thermoelectric device including thermoelectric elements having different sizes according to one embodiment.

FIGS. 17 and 18 are views for describing a thermoelectric element disposition pattern of the thermoelectric module including two pairs of terminals according to one embodiment.

### MODES OF THE INVENTION

Since embodiments described in the present specification are provided to clearly describe the spirit of the present invention to those skilled in the art, the present invention is not limited by the embodiments described in the present specification, and it should be understood that the scope of the present invention includes changes or modifications not departing from the spirit of the present invention.

Widely used general terms are selected as terms used in the present specification as much as possible in consideration of functions in the present invention, but may vary according to an intention of those skilled in the art, a custom, or emergence of new technology. However, when a specific term is defined as an arbitrary meaning and used, the meaning of the term will be separately disclosed. Accordingly, the terms used in this specification should be interpreted based on actual meanings of the terms and the contents throughout the present specification, instead of only names of the terms.

The accompanying drawings in the present specification are provided to easily describe the present invention, and since shapes shown in the drawings may be exaggerated as necessary to help understanding of the present invention, the present invention is not limited by the drawings.

In the description of the present disclosure, when it is determined that detailed descriptions of known configuration or functions related to the present invention may unnecessarily obscure the principle of the present invention, the detailed descriptions thereof will be omitted.

One aspect of the present invention provides a thermoelectric device including a plurality of thermoelectric groups connected in series between a first terminal and a second terminal, the first terminal and the second terminal provided in a plate shape deformable to a curved surface, having a first surface and a second surface opposite the first surface as a main surface, and configured to receive power from the outside, including wherein the plurality of thermoelectric groups include, a first thermoelectric group configured to provide any one of a feeling of coldness and a feeling of warmth toward the first surface when the power is applied, and including an N-type semiconductor and a P-type semiconductor disposed between the first surface and the second surface, and an electrode configured to alternately connect an N-type semiconductor and a P-type semiconductor and disposed adjacent to any one surface of the main surface, and a second thermoelectric group providing the other of the feeling of coldness and the feeling of warmth toward the first surface when the power is applied, and including an N-type semiconductor and a P-type semiconductor disposed between the first surface and the second surface, and an electrode configured to alternately connect an N-type semiconductor and a P-type semiconductor and disposed adjacent to any one surface of the main surfaces, wherein one end electrically adjacent to the first terminal of an electrode disposed adjacent to the first surface among the electrode of the second thermoelectric group is connected to the P-type semiconductor, when one end electrically adjacent to the first terminal of an electrode disposed adjacent to the first surface among the electrode of the first thermoelectric group is connected to the N-type semiconductor, wherein the one end electrically adjacent to the first terminal of the electrode disposed adjacent to the first surface among the electrode of the second thermoelectric group is connected to the N-type semiconductor, when the one end electrically adjacent to the first terminal of the electrode disposed adjacent to the first surface among the electrode of the first thermoelectric group is connected to the P-type semiconductor, and wherein the first thermoelectric group and the second thermoelectric group are connected through a connection electrode, and the connection electrode connects the N-type semiconductor of the first thermoelectric group and the N-type semiconductor of the second thermoelectric group or connects the P-type semiconductor of the first thermoelectric group and the P-type semiconductor of the second thermoelectric group.

Here, a number of N-type semiconductors and P-type semiconductors included in the first thermoelectric group may be greater than the number of N-type semiconductors and P-type semiconductors included in the second thermoelectric group.

Here, a ratio between the number of N-type semiconductors and P-type semiconductors included in the first thermoelectric group and the number of N-type semiconductors and P-type semiconductors included in the second thermoelectric group may be 0.2 to 4.

Here, a thermoelectric group including a greater number of N-type semiconductors and P-type semiconductors among the first thermoelectric group and the second thermoelectric group may provide a feeling of warmth toward the first surface.

Here, the first surface may include a plurality of thermal stimulus providing regions respectively corresponding to the plurality of thermoelectric groups, and wherein, an area of the thermal stimulus providing region corresponding to the first thermoelectric group may be larger than an area of the thermal stimulus providing region corresponding to the second thermoelectric group.

Here, a ratio between the area of the thermal stimulus providing region corresponding to the first thermoelectric group and the area of the thermal stimulus providing region corresponding to the second thermoelectric group may be 0.2 to 4.

Here, a thermoelectric group having a larger area of the corresponding thermal stimulus providing region among the first thermoelectric group and the second thermoelectric group may provide a feeling of warmth toward the first surface.

Here, each of the N-type semiconductors and the P-type semiconductors included in the plurality of thermoelectric groups are provided in a pillar shape having a bottom surface and a height, and areas of the bottom surfaces of the N-type semiconductors and the P-type semiconductors of the thermoelectric group providing the feeling of warmth toward the first surface among the plurality of thermoelectric groups may be larger than areas of the bottom surfaces of the N-type semiconductors and the P-type semiconductors of the thermoelectric group providing a feeling of coldness.

Here, a ratio between the areas of the bottom surfaces of the N-type semiconductors and the P-type semiconductors of the thermoelectric group providing the feeling of warmth toward the first surface among the plurality of thermoelectric groups and the areas of the bottom surfaces of the N-type semiconductors and the P-type semiconductors of the thermoelectric group providing the feeling of coldness may be 0.2 to 4.

Here, the thermoelectric device may provide a thermal grill illusion to a user through the first surface.

Here, the thermoelectric device may further include a heat dissipation sheet disposed on the second surface.

Here, heat dissipated toward the second surface by the thermoelectric group providing the feeling of coldness toward the first surface among the plurality of thermoelectric groups may be transferred to the thermoelectric group providing the feeling of warmth toward the first surface among the plurality of thermoelectric groups through the heat dissipation sheet.

Here, the first surface may include the plurality of thermal stimulus providing regions respectively corresponding to the plurality of thermoelectric groups, and an area of the thermal stimulus providing region corresponding to the thermoelectric group providing the feeling of warmth toward the first surface among the plurality of thermoelectric groups may be larger than an area of the thermal stimulus providing region corresponding to the thermoelectric group providing the feeling of coldness toward the first surface among the plurality of thermoelectric groups.

Here, a ratio between the area of the thermal stimulus providing region corresponding to the thermoelectric group providing the feeling of warmth toward the first surface among the plurality of thermoelectric groups and the area of the thermal stimulus providing region corresponding to the thermoelectric group providing the feeling of coldness toward the first surface among the plurality of thermoelectric groups may be 0.2 to 4.

Hereinafter, a thermoelectric module according to one embodiment and a thermoelectric device including the same will be described.

The thermoelectric device according to one embodiment is a device providing a thermal stimulus to a user. Specifically, the thermoelectric device may provide the thermal stimulus to the user by performing an exothermic operation or an endothermic operation to apply heat to the user or absorb heat from the user.

The thermal stimulus stimulates a user’s thermal sensory organs mainly distributed in the user’s body to cause the user to feel a thermal sensation, and in the present specification, it should be understood that the thermal stimulus encompasses everything that stimulates the user’s thermal sensory organs.

A feeling of warmth and a feeling of coldness may be representative examples of thermal stimulation. The feeling of warmth refers to applying warm heat to a hot spot distributed on the skin so that the user feels the feeling of warmth, and the feeling of coldness refers to applying cold heat to a cold spot distributed on the skin so that the user feels the feeling of coldness.

Here, since the heat is a physical quantity expressed in a positive scalar form, an expression ‘applying cold heat’ may not be a strict expression from a physical point of view, but in the present specification, for convenience of description, a phenomenon in which the heat is applied will be expressed as a case in which warm heat is applied, and a reverse phenomenon, that is, a phenomenon in which the heat is absorbed will be expressed as a case in which cold heat is applied.

Further, in the present specification, the thermal stimulus may further include a thermal grill illusion (TGI) in addition to the feeling of warmth and the feeling of coldness. The user perceives warm heat and cold heat as a sensation instead of individually perceiving the feeling of warmth and the feeling of coldness when the warmth and cold feelings are simultaneously given, and this sensation is referred to as the thermal grill illusion. That is, the thermal grill illusion refers to a thermal stimulus in which the hot heat and the cold heat are complexly applied, and may be mainly provided by simultaneously outputting the feeling of warmth and the feeling of coldness. Further, the thermal grill illusion may be referred to as a sensation of heat in an aspect of providing a sensation close to pain.

The thermal grill illusion may include a neutral thermal grill illusion, a warm thermal grill illusion, and a cold thermal grill illusion.

Here, the neutral thermal grill illusion, the warm thermal grill illusion, and the cold thermal grill illusion respectively cause a neutral thermal sensation, a warm thermal sensation, and a cold thermal sensation to the user. The neutral thermal sensation may mean that only a sensation is felt without a feeling of warmth or a feeling of coldness, the warm thermal sensation may mean that a sensation is felt in addition to the feeling of warmth, and the cold thermal sensation may mean that a sensation is felt in addition to the feeling of coldness.

The neutral thermal sensation is caused when intensity of the user’s feelings of warmth and coldness corresponds to a predetermined rate range. A rate of feeling the neutral thermal sensation (hereinafter, referred to as ‘a neutral rate’) may be different for each body part which receives a thermal stimulus, and may be slightly different for each individual even in the case of the same body part, and in most cases, there is a tendency in that the neutral thermal sensation is felt in a situation in which the intensity of the feeling of coldness is greater than the intensity of the feeling of warmth. The warm thermal sensation may be felt when the magnitude of the feeling of warmth is greater than the neutral rate, and the cold thermal sensation may be felt when the magnitude of the feeling of coldness is greater than the neutral rate.

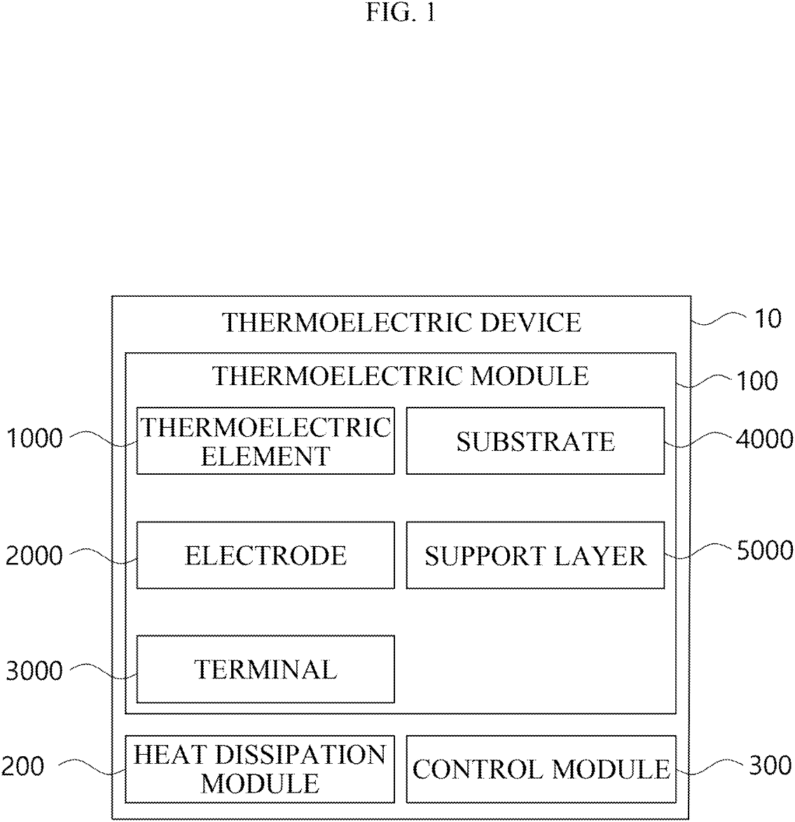

FIG. 1 is a view related to a configuration of a thermoelectric device according to one embodiment. Referring to FIG. 1, a thermoelectric device 10 according to one embodiment may include a thermoelectric module 100, a heat dissipation module 200, and a control module 300. In the thermoelectric device 10, the control module 300 may provide a thermal stimulus to a user by controlling the thermoelectric module 100 to selectively or simultaneously perform an exothermic operation or an endothermic operation, and waste heat due to a thermoelectric operation of the thermoelectric module 100 may be discharged to the outside of the thermoelectric device 10 through the heat dissipation module 200.

The thermoelectric device according to one embodiment may include a thermoelectric module.

The thermoelectric module may refer to a module which performs a power generation operation using a temperature difference using a thermoelectric effect such as the Seebeck effect, the Peltier effect, or the like or performs a thermoelectric operation of a heating operation, a cooling operation, or the like using electric energy. For example, the thermoelectric module may provide a thermal stimulus including a feeling of warmth, a feeling of coldness, and a thermal grill illusion to the user through the thermoelectric operation.

Referring to FIG. 1, the thermoelectric module 100 according to one embodiment may include thermoelectric elements 1000, electrodes 2000, terminals 3000, substrates 4000, and support layers 5000.

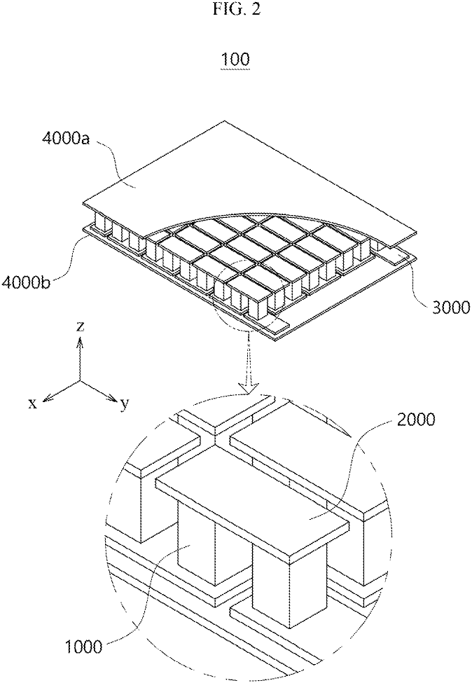

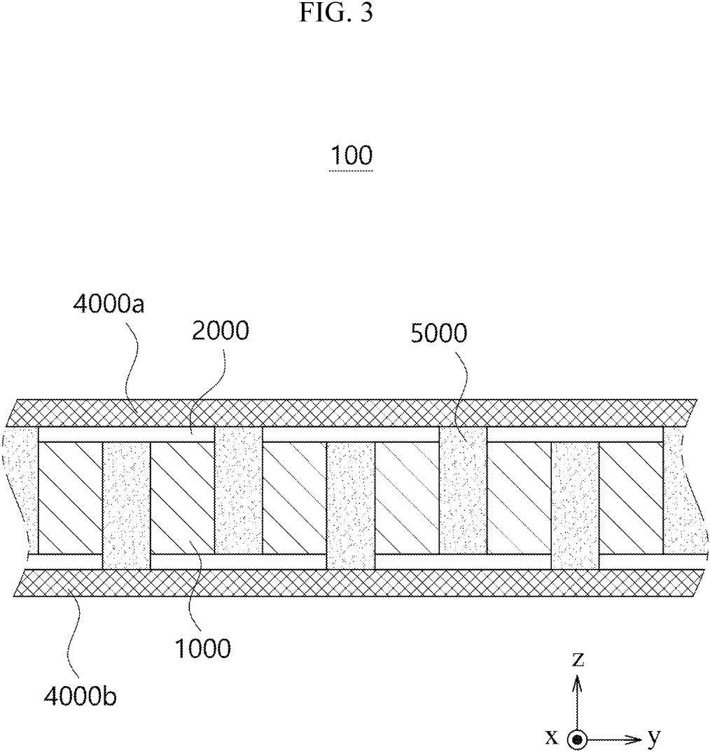

FIG. 2 is a perspective view related to the thermoelectric module according to one embodiment, and FIG. 3 is a cross-sectional view related to the thermoelectric module according to one embodiment. Hereinafter, the thermoelectric elements 1000 will be described with reference to FIGS. 2 and 3.

The thermoelectric module 100 may be provided in a plate shape. Further, the thermoelectric module 100 may have flexibility (hereinafter, referred to as “a flexible thermoelectric module”). The flexible thermoelectric module 100 is basically provided in the plate shape, but has flexibility in which curving is possible, and thus may be deformed into various shapes including a curved shape. Further, the flexible thermoelectric module 100, which may be deformed between a flat shape and a curved shape, may be provided. For example, the flexible thermoelectric module 100 of a flat shape as shown in FIG. 2 may be deformed into a curved shape by applying a force, and when the applied force is removed, the flexible thermoelectric module 100 in the curved shape may be restored to the flat shape.

The thermoelectric elements 1000 may be elements which cause a thermoelectric effect such as the Seebeck effect, the Peltier effect, or the like. Basically, the thermoelectric elements 1000 may include a first thermoelectric element and a second thermoelectric element of different materials constituting a thermoelectric pair (thermoelectric couple) which causes the thermoelectric effect. The first thermoelectric element and the second thermoelectric element are electrically connected to each other to form a thermoelectric pair. The thermoelectric pair may generate a temperature difference when electrical energy is applied, and on the other hand, may produce electrical energy when the temperature difference is applied. A pair of bismuth telluride (Bi-Te) and antimony telluride (Sb-Te) is a representative example of the thermoelectric element 1000. Further, recently, a pair of an N-type semiconductor and a P-type semiconductor is mainly used as the thermoelectric element 1000.

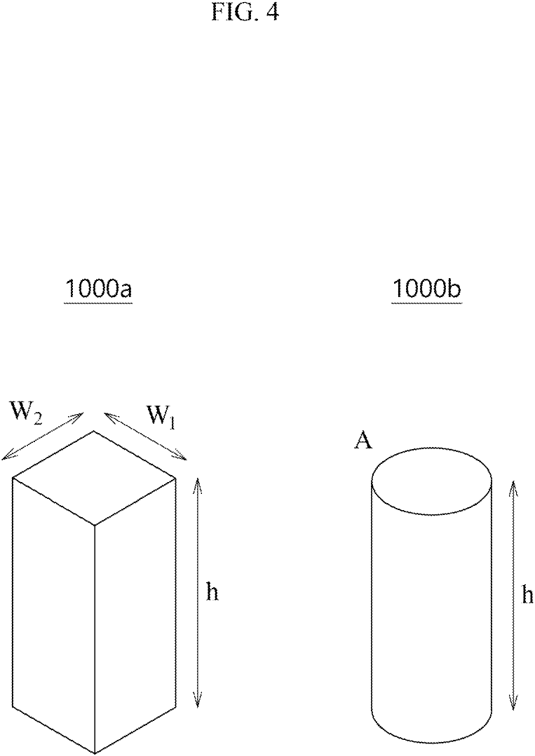

The thermoelectric element 1000 may be provided in a pillar shape. FIG. 4 is a view related to thermoelectric elements according to one embodiment. Referring to FIG. 4, the pillar-shaped thermoelectric element 1000 may be expressed by a bottom surface and a height. For example, a thermoelectric element 1000a having a quadrangular pillar shape may be expressed by a bottom surface including a width w1 and a length w2, and a height h. In this case, an area of the bottom surface of the thermoelectric element 1000a may be expressed as a product of the width and the length. As another example, a pillar-shaped thermoelectric element 1000b may be expressed by a bottom surface having an area A and a height h. In addition, the thermoelectric element 1000 may be provided in another shape such as a triangular pillar shape or the like.

The electrode 2000 electrically connects the thermoelectric elements 1000. The thermoelectric elements 1000 may generate the thermoelectric effect only when at least the first thermoelectric element and the second thermoelectric element of different materials are electrically connected to each other to form the thermoelectric pair. Accordingly, the electrode 2000 may form a thermoelectric pair by connecting the first thermoelectric element and the second thermoelectric element adjacent to each other. Of course, the electrode 2000 may electrically connect the thermoelectric elements 1000 of the same material, such as connecting the first thermoelectric element and the first thermoelectric element, connecting the second thermoelectric element and the second thermoelectric element, or the like.

Further, the electrode 2000 may connect a plurality of thermoelectric elements 1000 in series. The thermoelectric elements 1000 connected in series by the electrode 2000 may form a thermoelectric group which simultaneously performs the same thermoelectric operation.

The electrode 2000 may be provided with a metal material such as copper, silver, or the like, but the present invention is not limited thereto.

FIG. 2 illustrates that the electrodes 2000 are disposed in a form of covering both upper surfaces and lower surfaces of the thermoelectric elements 1000, but a size relationship between the electrodes 2000 and the thermoelectric elements 1000 is not limited thereto, and the electrodes 2000 may be disposed in a form of covering only portions of the upper surfaces or the lower surfaces of the thermoelectric elements 1000.

The terminals 3000 are terminals that connect the thermoelectric module 100 to the outside. For example, the thermoelectric module 100 may be connected to the control module 300 through the terminals 3000.

When the thermoelectric module 100 is used as a heat outputting module, the terminals 3000 may supply power for performing a heating/cooling operation using the Peltier effect by the thermoelectric module 100. Further, when the thermoelectric module 100 is used as a thermoelectric generating module, the terminal 3000 may transfer power generated by the thermoelectric module 100 using the Seebeck effect to the outside.

A pair of substrates 4000a and 4000b may be disposed to be spaced apart from each other to face each other. The substrates 4000 may support the thermoelectric elements 1000 or the electrodes 2000 disposed therebetween. Further, the substrates 4000 may perform a function of protecting the thermoelectric elements 1000 or the electrodes 2000 therein from the outside.

Each of the substrates 4000 may be provided with a material which easily conducts heat and has flexibility. For example, the substrate 4000 may be a thin polyimide (PI) film. The polyimide film may have excellent flexibility and may be manufactured in a thin thickness although thermal conductivity is not high, and thus may be advantageous for conducting heat.

The substrate 4000 may be disposed on only one surface of the thermoelectric module 100. The flexible thermoelectric module 100 having the substrate 4000 on only one surface has an advantage of having improved flexibility compared to the flexible thermoelectric module 100 having the substrate 4000 on both surfaces because the substrate 4000 has some resistance to curving or the like even when provided with a flexible material such as a PI film.

In the flexible thermoelectric module 100 having the substrate 4000 on only one surface, a surface not having the substrate 4000 thereon may have greater flexibility than an opposite surface. When the surface not having the substrate 4000 thereon is used as a convex portion while using the flexible thermoelectric module 100 as the curved shape, these advantages may be fully taken.

The thermoelectric module 100 may include the support layers 5000. The support layers 5000 may be located between the pair of substrates 4000a and 4000b. The support layers 5000 may support the thermoelectric elements 1000 and the electrodes 2000. Accordingly, the thermoelectric elements 1000 and the electrodes 2000 may be supported together with the substrates 4000 by the support layers 5000.

The support layers 5000 may be provided with a flexible material so that the thermoelectric module 100 may maintain flexibility. For example, the support layers 5000 may be foaming layers having internal pores like a sponge. Here, the foaming layers may be formed by filling a foaming agent between the pair of substrates 4000a and 4000b. An organic foaming agent, an inorganic foaming agent, a physical foaming agent, polyurethane, silicone foam, and the like may be used as the foaming agent.

When the support layers 5000 are included in the thermoelectric module 100, since the thermoelectric elements 1000 and the electrodes 2000 may be supported by the support layers 5000, the substrates 4000 may not be necessarily required. Any one substrate 4000 of the pair of substrates 4000a and 4000b shown in FIGS. 2 and 3 may be removed. Alternatively, both the pair of external substrates 4000a and 4000b may be removed. When the substrates 4000 are removed, the flexibility of the thermoelectric module 100 may be improved.

The thermoelectric module 100 in FIGS. 2 and 3 is only an example, and the thermoelectric module 100 according to one embodiment may be provided by excluding some components or adding additional components. For example, the thermoelectric module 100 according to one embodiment may not include at least one of the substrates 4000 and the support layers 5000.

The thermoelectric device according to one embodiment may include a heat dissipation module.

The heat dissipation module may dissipate heat absorbed from the thermoelectric module to the outside. For example, waste heat generated as the thermoelectric module operates may be transferred to the heat dissipation module, and the heat dissipation module may discharge the received waste heat to the outside.

The heat dissipation module may be implemented with a material having high thermal conductivity. For example, the heat dissipation module may be implemented using a metal material and/or an alloy material such as aluminum or magnesium. As another example, the heat dissipation module may be implemented using a thermally conductive polymer. In addition, the heat dissipation module may be implemented with various materials such as a ceramic, a carbon composite material, a polymer/metal composite material, a polymer/ceramic composite material, and the like.

In the heat dissipation module, one region is disposed to correspond to a high temperature region and the other region is disposed to correspond to a low temperature region. The heat dissipation module may be formed in a structure capable of increasing an area which comes into contact with the low temperature region. For example, in the heat dissipation module, the one region may be provided in a plate shape and the other region may be provided in a fin shape. Alternatively, the other region may be formed to have a protrusion and a recessed portion. Of course, both the one region and the other region of the heat dissipation module may each be provided in a plate shape.

The thermoelectric device according to one embodiment may include a control module.

The control module may control the overall operation of the thermoelectric device. For example, the control module may be connected to a terminal of the thermoelectric module to apply power to the thermoelectric element to control the thermoelectric module so that the thermoelectric module may perform the exothermic operation or the endothermic operation.

To this end, the control module may control the operation of the thermoelectric module by performing calculation and processing of various pieces of information and outputting an electrical signal to the thermoelectric module according to a processing result. Accordingly, the control module may be implemented as a computer or similar device according to hardware, software, or a combination thereof. In the hardware, the control module may be provided in the form of an electronic circuit which performs a control function by processing electrical signals, and in the software, the control module may be provided in the form of a program or code which drives a hardware circuit. In the following description, unless otherwise mentioned, it may be interpreted that the operation of the thermoelectric device is performed by the control of the control module.

Hereinafter, a connection relationship between the thermoelectric elements, the electrodes, and the terminals for providing the thermal stimulus to the user will be described.

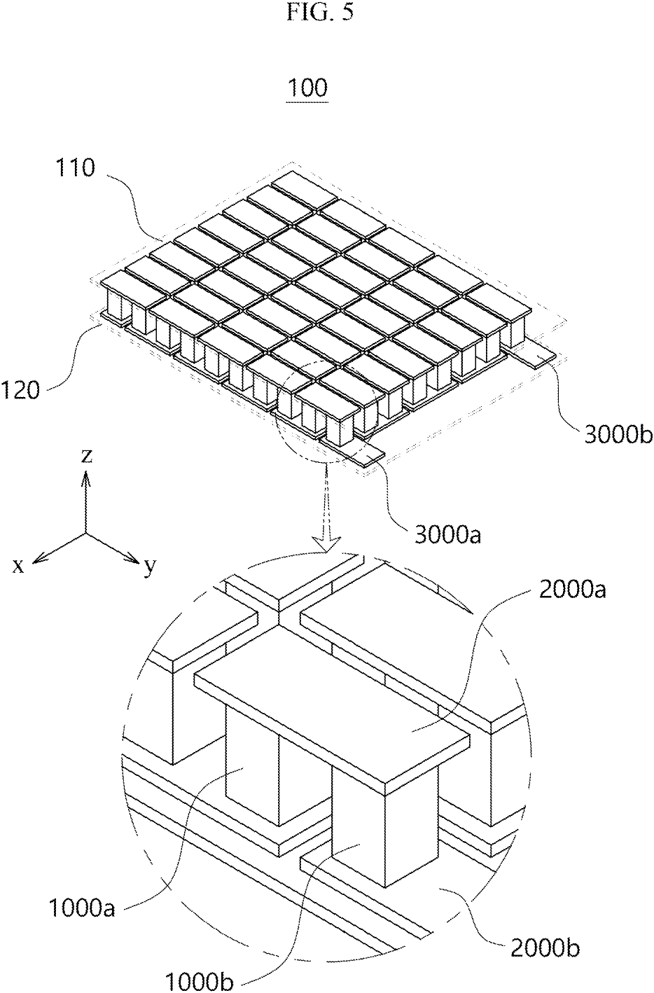

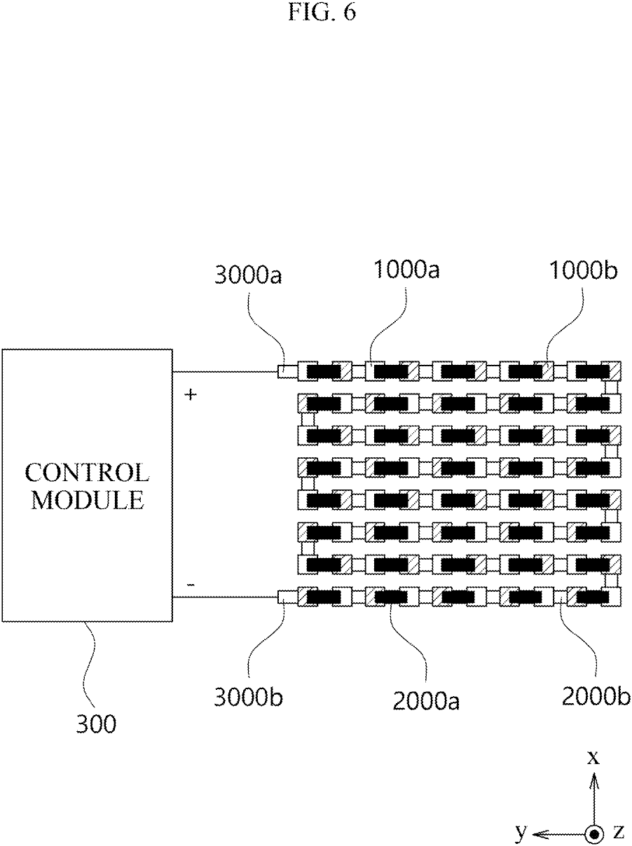

FIG. 5 is a perspective view related to a connection between the thermoelectric elements, the electrodes, and the terminals according to one embodiment, and FIG. 6 is a plan view related to the connection between the thermoelectric elements, the electrodes, and the terminals according to one embodiment. Referring to FIGS. 5 and 6, the thermoelectric module 100 according to one embodiment may include the thermoelectric elements 1000 connected through the electrodes 2000 between a pair of terminals 3000.

The thermoelectric module 100 according to one embodiment may include a first surface 110 and a second surface 120 that are two main surfaces facing each other. Hereinafter, a direction of the first surface 110 is expressed as an upper surface and a direction of the second surface 120 is expressed as a lower surface, but this is for convenience of explanation, and this expression does not limit a disposition position or method of the thermoelectric module 100.

The thermoelectric module 100 according to one embodiment may include different types of thermoelectric elements 1000 that are alternately connected. As described above, the thermoelectric elements 1000 may include first thermoelectric elements 1000a and second thermoelectric elements 1000b constituting a thermoelectric pair, and the thermoelectric module 100 according to one embodiment may include the first thermoelectric elements 1000a and the second thermoelectric elements 1000b that are alternately connected.

The thermoelectric elements 1000 may be electrically connected through the electrodes 2000. For example, the first thermoelectric element 1000a and the second thermoelectric element 1000b may be connected through the electrode 2000.

The electrodes 2000 may include first electrodes 2000a disposed adjacent to the first surface 110 and second electrodes 2000b disposed adjacent to the second surface 120. As shown in FIG. 5, the first electrode 2000a may be disposed on upper surfaces of the first thermoelectric element 1000a and the second thermoelectric element 1000b to connect the first thermoelectric element 1000a and the second thermoelectric element 1000b, and the second electrode 2000b may be disposed on lower surfaces of the first thermoelectric element 1000a and the second thermoelectric element 1000b to connect the first thermoelectric element 1000a and the second thermoelectric element 1000b.

The first electrodes 2000a and the second electrodes 2000b may alternately connect the first thermoelectric elements 1000a and the second thermoelectric elements 1000b. For example, the first thermoelectric element 1000a, the first electrode 2000a, the second thermoelectric element 1000b, and the second electrode 2000b may be connected in this order. Alternatively, the first thermoelectric element 1000a, the second electrode 2000b, the second thermoelectric element 1000b, and the first electrode 2000a may be connected in this order.

The thermoelectric module 100 according to one embodiment may include the terminals 3000 which receive power from the outside. As shown in FIG. 6, one end of the terminal 3000 may be connected to the control module 300 and the other end may be connected to the thermoelectric elements 1000.

The pair of terminals 3000a and 3000b may respectively receive power of a (+) polarity and power of a (-) polarity. Here, the polarities of the power applied to the terminals 3000 may be changed. For example, the (-) power may be applied to a terminal to which the (+) power is applied, and vice versa.

FIG. 6 illustrates that the electrodes 2000a and 2000b are disposed in a form of covering only portions of the upper surfaces or the lower surfaces of the thermoelectric elements 1000a and 1000b, but the size relationship between the electrodes 2000a and 2000b and the thermoelectric elements 1000a and 1000b is not limited thereto, and the electrodes 2000a and 2000b may be disposed in a form of covering both the upper surfaces or the lower surfaces of the thermoelectric elements 1000a and 1000b.

In the case of the thermoelectric module 100 in which the first thermoelectric element 1000a and the second thermoelectric element 1000b of different materials are alternately connected, a feeling of warmth or a feeling of coldness may be provided to the user in one direction.

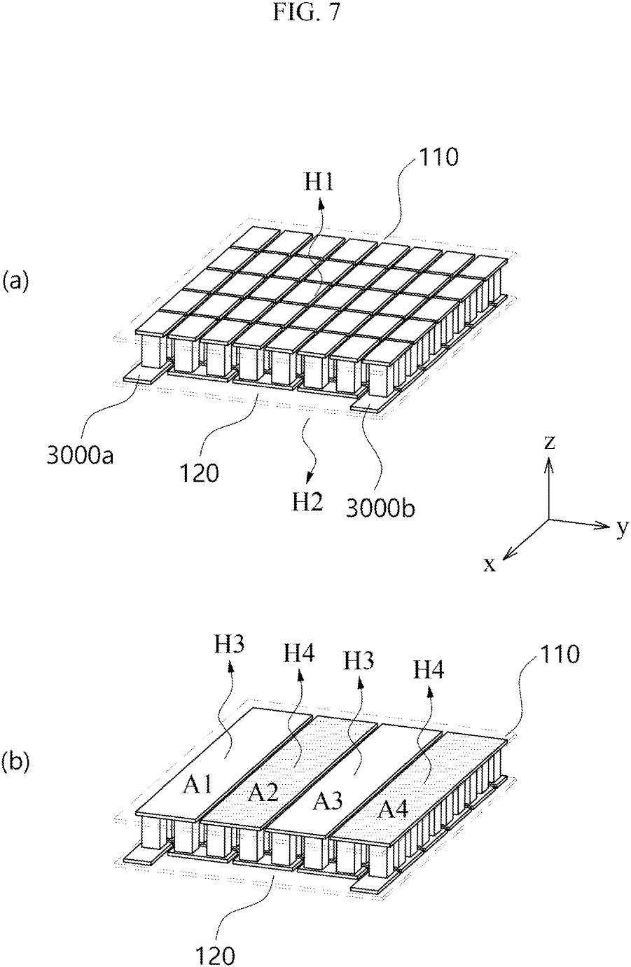

FIG. 7 is a view related to the thermoelectric module providing a thermal stimulus according to one embodiment. Referring to FIGS. 6 and 7A, when the (+) power is applied to the first terminal 3000a and the (-) power is applied to the second terminal 3000b, a feeling of warmth or a feeling of coldness may be provided to the user through the first surface 110. For example, the (+) power may be applied to the first terminal 3000a and the (-) power may be applied to the second terminal 3000b to provide a feeling of warmth to the user through the first surface 110. In this case, when the (-) power is applied to the first terminal 3000a and the (+) power is applied to the second terminal 3000b, a feeling of coldness may be provided to the user through the first surface 110.

Hereinafter, for convenience of description, it is described that the thermoelectric module 100 is disposed so that the first surface 110 becomes adjacent to a user’s skin, but this is only for convenience of description, and the thermoelectric module 100 may be disposed so that the second surface 120 may become adjacent to the user’s skin, and this expression does not limit the disposition position or method of the thermoelectric module 100.

Cold heat may be provided toward the second surface 120 when the thermoelectric module 100 provides warm heat toward the first surface 110, and conversely, warm heat may be provided toward the second surface 120 when the thermoelectric module 100 provides cold heat toward the first surface 110.

As described above, when the thermoelectric device simultaneously outputs the feeling of coldness and the feeling of warmth, the thermal grill illusion may be provided to the user. For example, referring to FIG. 7B, in the case in which a feeling of coldness and a feeling of warmth are simultaneously output through the first surface 110 such as a case in which a first region A1 and a third region A3 of the first surface 110 of the thermoelectric module provide any one of the feeling of warmth and the feeling of coldness, and a second region A2 and a fourth region A4 of the first surface 110 of the thermoelectric module provide the other of the feeling of warmth and the feeling of coldness or the like, the thermoelectric module may provide the thermal grill illusion to the user.

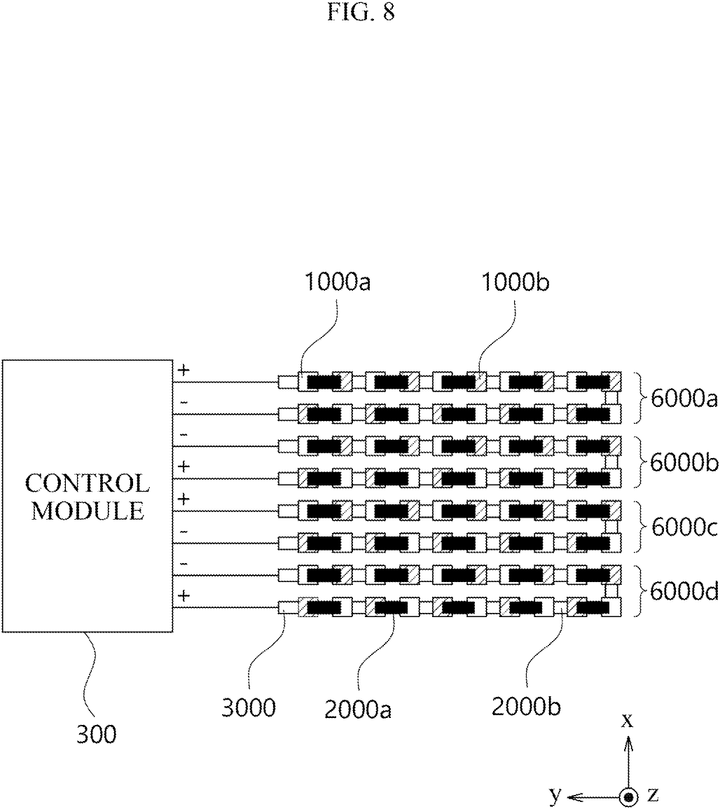

FIG. 8 is a view related to the thermoelectric module providing the thermal grill illusion according to one embodiment. When comparing FIGS. 6 and 8, when the thermoelectric elements 1000 are alternately connected to form a single thermoelectric group as shown in FIG. 6 and the thermoelectric module includes only a pair of terminals, since the thermoelectric module may output only one of a feeling of coldness and a feeling of warmth toward the first surface, a thermal grill illusion may not be provided to the user. On the other hand, in the case of FIG. 8, when the alternately connected thermoelectric elements 1000 form a plurality of thermoelectric groups 6000, and each thermoelectric group is connected to a pair of terminals and thus the thermoelectric module includes the same number of terminal pairs as the thermoelectric group, since the thermoelectric module may simultaneously output a feeling of coldness and a feeling of warmth, the thermal grill illusion may be provided to the user. For example, as shown in FIG. 8, in the plurality of thermoelectric groups 6000 that are alternately connected in the same order, when a direction of power applied to a first thermoelectric group 6000a and a third thermoelectric group 6000c becomes different from a direction of power applied to a second thermoelectric group 6000b and a fourth thermoelectric group 6000d, the first thermoelectric group 6000a and the third thermoelectric group 6000c may output any one of a feeling of warmth and a feeling of coldness and the second thermoelectric group 6000b and the fourth thermoelectric group 6000d may output the other one of the feeling of warmth and the feeling of coldness to provide a thermal grill illusion to the user.

However, when the thermoelectric device is configured as shown in FIG. 8, since a pair of terminals may be connected to each thermoelectric group, a size and a weight of the thermoelectric device may increase. Additional means for individual control of the thermoelectric group may also be required.

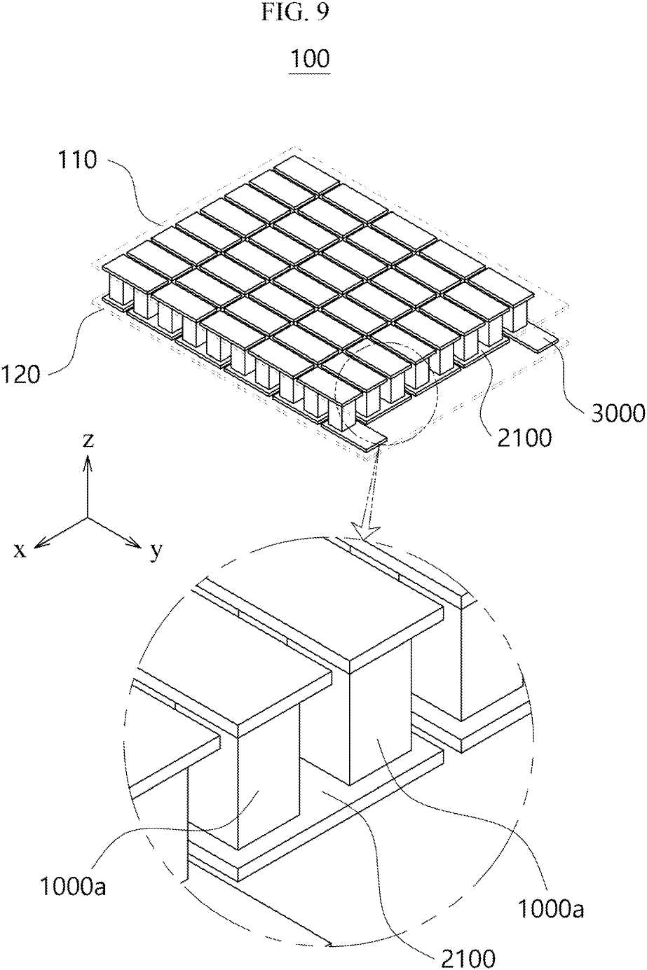

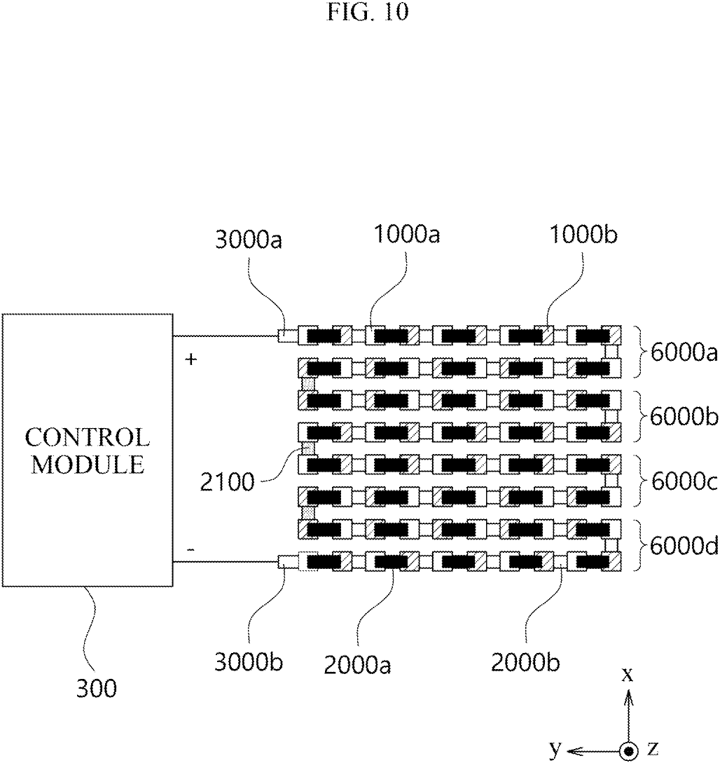

FIG. 9 is a perspective view related to the thermoelectric module providing the thermal grill illusion according to one embodiment, and FIG. 10 is a plan view related to the thermoelectric module providing the thermal grill illusion according to one embodiment. Referring to FIGS. 9 and 10, the thermoelectric module 100 according to one embodiment may include the plurality of thermoelectric groups 6000 including the first thermoelectric elements 1000a, the second thermoelectric elements 1000b, and the electrodes 2000.

The first thermoelectric elements 1000a and the second thermoelectric elements 1000b included in the same thermoelectric group 6000 may be alternately connected through the electrodes 2000. Further, the thermoelectric elements 1000 included in different thermoelectric groups 6000 may also be connected through the electrodes 2000. Hereinafter, the electrodes 2000 which connect the thermoelectric elements 1000 in the thermoelectric group 6000 are referred to as general electrodes, and the electrodes 2000 which connect the thermoelectric elements 1000 included in different thermoelectric groups 6000 are referred to as connection electrodes 2100, but this is for convenience of description, and does not mean that materials or shapes of the general electrodes and the connection electrodes 2100 should be different.

When comparing FIGS. 6 and 10, in the thermoelectric module in FIG. 6, the first thermoelectric elements 1000a and the second thermoelectric elements 1000b may be alternately connected through the electrodes 2000 between the pair of terminals 3000. On the other hand, in the case of FIG. 10, the thermoelectric module includes a pair of terminals 3000, and the first thermoelectric element 1000a and the second thermoelectric element 1000b are alternately connected through the electrodes 2000 in each thermoelectric group like FIG. 6, but when the thermoelectric elements 1000 included in the different thermoelectric groups 6000 are connected through the connection electrode 2100, the same type of thermoelectric elements 1000 are connected. For example, as shown in FIG. 10, the second thermoelectric element 1000b of the first thermoelectric group 6000a and the second thermoelectric element 1000b of the second thermoelectric group 6000b may be connected through the connection electrode 2100, the first thermoelectric element 1000a of the second thermoelectric group 6000b and the first thermoelectric element 1000a of the third thermoelectric group 6000c may be connected through the connection electrode 2100, and the second thermoelectric element 1000b of the third thermoelectric group 6000c and the second thermoelectric element 1000b of the fourth thermoelectric group 6000d may be connected through the connection electrode 2100.

Types of thermoelectric elements connected to both ends of the electrode may vary according to the thermoelectric group as the same type of thermoelectric elements included in different thermoelectric groups are connected. As described above, when the electrodes disposed adjacent to the first surface of the thermoelectric module are referred to as the first electrodes 2000a and the electrodes disposed adjacent to the second surface are referred to as the second electrodes 2000b, referring to FIG. 10, in the case in which the first thermoelectric element 1000a is connected to one end of the first electrode 2000a included in the first thermoelectric group 6000a that is electrically adjacent to the first terminal 3000a, and the second thermoelectric element 1000b is connected to the other end of the first electrode 2000a, the second thermoelectric element 1000b may be connected to one end of the first electrode 2000a included in the second thermoelectric group 6000b connected to the first thermoelectric group 6000a that is electrically adjacent to the first terminal 3000a, and the first thermoelectric element 1000a may be connected to the other end of the first electrode 2000a. Here, those skilled in the art may understand that a meaning of being electrically adjacent includes a conceptual meaning in that a current, which starts at a specific point, arrives first in view of a current flow. For example, as described above, a case in which the first thermoelectric element is connected to one end of the first electrode electrically adjacent to the first terminal and the second thermoelectric element is connected to the other end of the first electrode may mean that a current that starts from the first terminal flows in an order of the first thermoelectric element, one end of the first electrode, the other end of the first electrode, and the second thermoelectric element.

When the same type of thermoelectric elements included in different thermoelectric groups are connected, the different thermoelectric groups may output different thermal stimuli. Referring to FIG. 10, the first thermoelectric group 6000a and the third thermoelectric group 6000c may output any one of a feeling of warmth and a feeling of coldness, and the second thermoelectric group 6000b and the fourth thermoelectric group 6000d may output the other one of the feeling of warmth and the feeling of coldness. As a result, the thermoelectric module may provide the thermal grill illusion to the user while including only a pair of terminals.

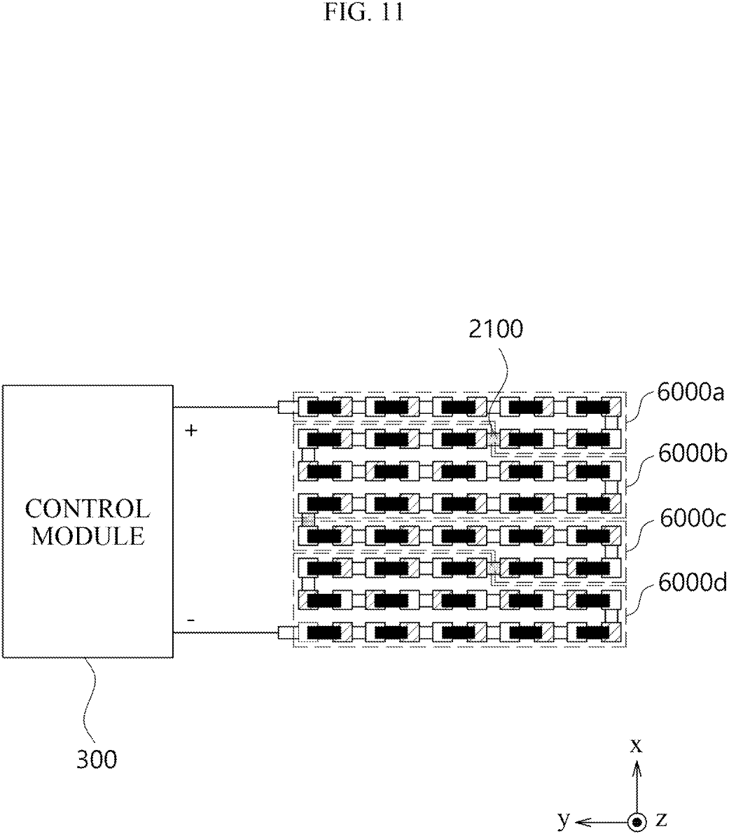

FIG. 11 is a plan view related to another example of the thermoelectric module providing the thermal grill illusion according to one embodiment. Hereinafter, the thermoelectric module will be described by comparing FIGS. 10 and 11.

The number of thermoelectric elements included in the thermoelectric group may vary for each thermoelectric group. For example, in the case of FIG. 10, each thermoelectric group 6000 includes 20 thermoelectric elements 1000, but as shown in FIG. 11, the number of thermoelectric elements included in the thermoelectric group may vary depending on the case, and for example, the first thermoelectric group 6000a may include 14 thermoelectric elements 1000 and the second thermoelectric group 6000b may include 26 thermoelectric elements, and even thermoelectric groups providing the same thermal stimulus may include different numbers of thermoelectric elements.

An area of the region where the thermoelectric group provides the thermal stimulus to the user (hereinafter, referred to as the “thermal stimulus providing region”) may vary for each thermoelectric group. For example, in the case of FIG. 10, the thermal stimulus providing regions of all thermoelectric groups 6000a, 6000b, 6000c, and 6000d have the same size, but in the case of FIG. 11, the second thermoelectric group 6000b and the fourth thermoelectric group 6000d have larger thermal stimulus providing regions compared to the first thermoelectric group 6000a and the third thermoelectric group 6000c.

The area of the thermal stimulus providing region of the thermoelectric group may vary according to the type of thermal stimulus provided to the user by the thermoelectric group. For example, the size of the thermal stimulus providing region of the thermoelectric group which outputs warm heat to the user may be different from the size of the thermal stimulus providing region of the thermoelectric group which outputs cold heat. Intensity or feeling of the stimulus felt by the user may vary due to a size difference between the thermal stimulus providing regions according to the types of the thermal stimuli.

The type of thermal grill illusion provided to the user by the thermoelectric module may vary by adjusting the area of the thermal stimulus providing region. For example, when the thermoelectric module provides a neutral thermal grill illusion in the case in which a size of the thermal stimulus providing region of the thermoelectric group providing a feeling of warmth and a size of the thermal stimulus providing region of the thermoelectric group providing a feeling of coldness are provided in a predetermined ratio, the thermoelectric module may provide a warm thermal grill illusion when the size of the thermal stimulus providing region of the thermoelectric group providing the feeling of warmth increases, and the thermoelectric module may provide a cold thermal grill illusion when the size of the thermal stimulus providing region of the thermoelectric group providing the feeling of coldness increases.

Positions of the connection electrodes may vary according to the thermoelectric module. For example, in the case of FIG. 10, the connection electrodes 2100 are disposed at a left region of the thermoelectric module, but a disposition position of the connection electrodes is not limited thereto, and as shown in FIG. 11, the connection electrodes 2100 may be disposed to connect the thermoelectric elements at an arbitrary position in the thermoelectric module such as a central region of the thermoelectric module or the like.

A case in which the thermoelectric device provides the thermal grill illusion may be advantageous for heat dissipation compared to a case in which the feeling of warmth and the feeling of coldness are provided.

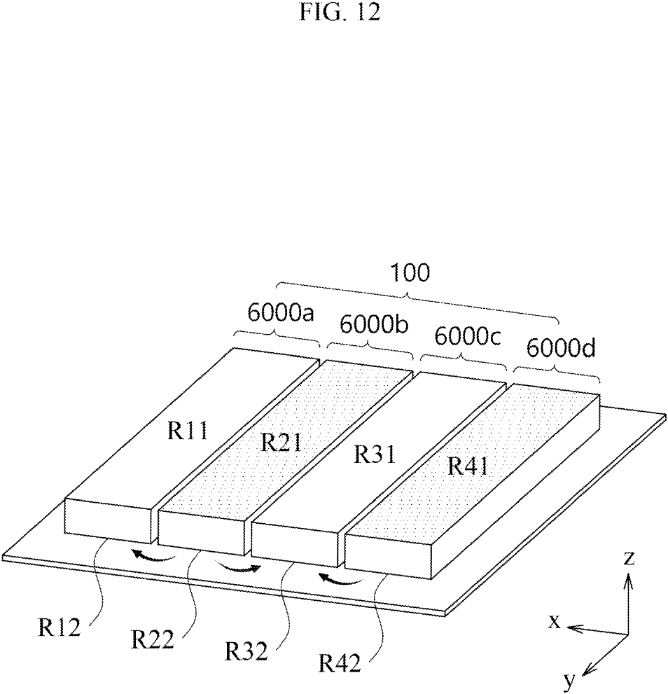

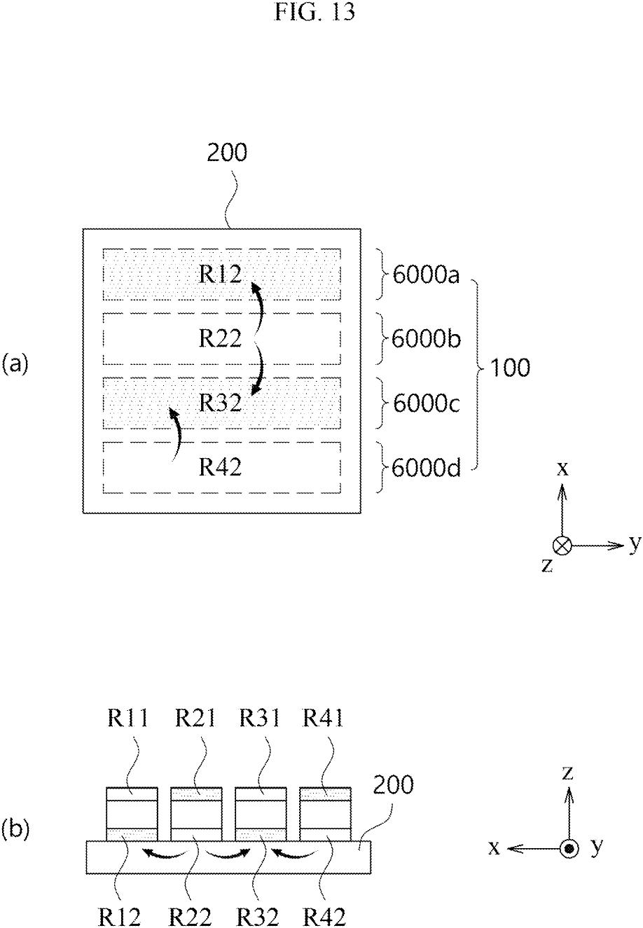

FIG. 12 is a perspective view related to a heat flow of a thermoelectric device providing a thermal grill illusion according to one embodiment, and FIG. 13 shows a rear view and a front view related to the heat flow of the thermoelectric device providing the thermal grill illusion according to one embodiment. Referring to FIGS. 12 and 13, the thermoelectric device providing the thermal grill illusion according to one embodiment may include the thermoelectric module 100 including the plurality of thermoelectric groups 6000 and the heat dissipation module 200. Hereinafter, for convenience of description, it is considered that the thermoelectric module 100 provides the thermal stimulus to the user through the first surface and the heat dissipation module 200 is disposed on the second surface.

The thermoelectric group may output any one of warm heat and cold heat in one direction and may output the other of the warm heat and the cold heat in the other direction by receiving the power and performing a thermoelectric operation. For example, referring to FIGS. 12 and 13, the thermoelectric groups 6000 may include first regions R11, R21, R31, and R41 which output any one of the warm heat and the cold heat toward the first surface and second regions R12, R22, R32, and R42 which output the other of the warm heat and the cold heat toward the second surface. Here, the first regions R11, R21, R31, and R41 may be regarded as the thermal stimulus providing regions.

Some of the plurality of thermoelectric groups may provide the warm heat toward the first surface and the remaining thermoelectric groups may provide the cold heat toward the first surface. In this case, the thermoelectric groups providing the warm heat toward the first surface may include a first region which outputs the warm heat and a second region which outputs the cold heat, and the thermoelectric groups providing the cold heat toward the first surface may include a first region which outputs the cold heat and a second region which outputs the warm heat. For example, referring to FIGS. 12 and 13, the first thermoelectric group 6000a and the third thermoelectric group 6000c may include the first regions R11 and R31 which output the warm heat to provide the warm heat toward the first surface and the second regions R12 and R32 which output the cold heat, and the second thermoelectric group 6000b and the fourth thermoelectric group 6000d may include the first regions R21 and R41 which output the cold heat to provide the cold heat toward the first surface and the second regions R22 and R42 which output the warm heat. As a result, since the thermoelectric module 100 simultaneously provides the cold heat and the warm heat to the user through the first surface, the user may feel the thermal grill illusion.

The warm heat output from the thermoelectric group may be transferred to the thermoelectric group adjacent thereto through the heat dissipation module. Specifically, when the thermoelectric group which outputs the warm heat and the thermoelectric group which outputs the cold heat are adjacent to each other, the warm heat output from the thermoelectric group which outputs the warm heat may be transferred to the thermoelectric group which outputs the cold heat. For example, referring to FIGS. 12 and 13, the warm heat output from the second region R22 of the second thermoelectric group 6000b may be transferred to the second regions R12 and R32 of the first thermoelectric group 6000a and the third thermoelectric group 6000c through the heat dissipation module 200, and the warm heat output from the second region R42 of the fourth thermoelectric group 6000d may be transferred to the second region R32 of the third thermoelectric group 6000c through the heat dissipation module 200. In this case, since the second regions R12 and R32 of the first thermoelectric group 6000a and the third thermoelectric group 6000c output the cold heat, the second regions R12 and R32 of the first thermoelectric group 6000a and the third thermoelectric group 6000c have lower temperatures than the second regions R22 and R42 of the second thermoelectric group 6000b and the fourth thermoelectric group 6000d, and thus may easily receive the heat transferred from the second regions R22 and R42 of the second thermoelectric group 6000b and the fourth thermoelectric group 6000d.

For this heat transfer structure, the heat dissipation sheet may be a single sheet. In this case, the plurality of thermoelectric groups may be disposed on one surface of the single sheet. Further, the other surface (a surface opposite the one surface) of the single sheet is formed as a flat surface. For example, the single sheet may have a finless configuration of a heat sink, and the one surface and the other surface of the single sheet may be flat surfaces. In this case, when the single sheet is bent, the flat surface may be a curved surface of which an outer surface is flat.

Further, the single sheet may have a thickness of 0.1 to 20 millimeters. More specifically, the single sheet may have a thin thickness of 0.1 to 3 millimeters.

In the case of the thermoelectric device providing the thermal grill illusion by heat transfer between the thermoelectric groups, heat dissipation may be easier compared to a thermoelectric device providing a feeling of warmth and/or a feeling of coldness. A general thermoelectric device may use a large heat dissipation module such as a heat sink including a fin-shaped structure or the like for heat dissipation, but the thermoelectric device providing the thermal grill illusion may provide a sufficient heat dissipation effect using only a sheet-shaped heat dissipation module without including a fin structure.

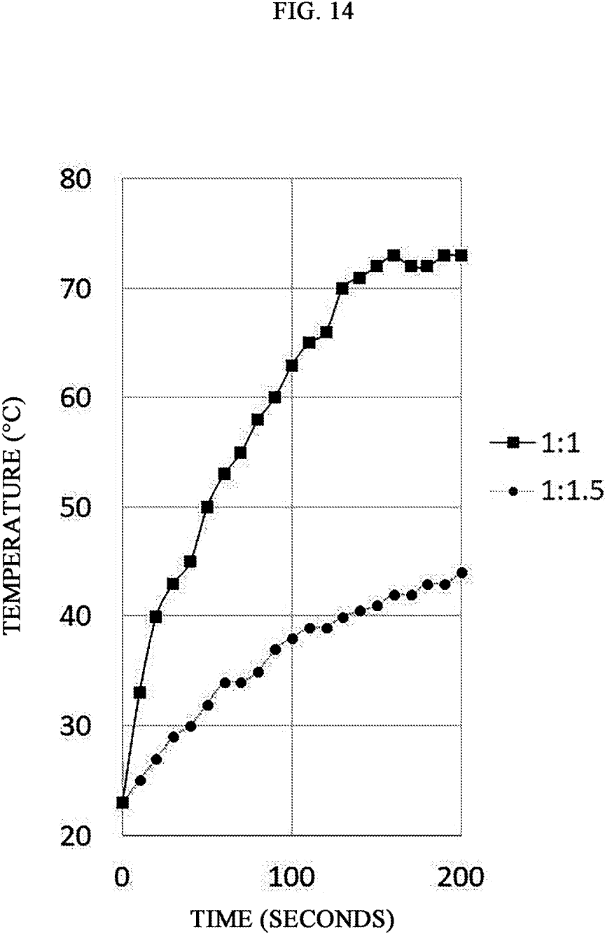

FIG. 14 is a graph illustrating a temperature change of the heat dissipation module according to one embodiment. Data expressed as circles is a temperature of the heat dissipation module of the thermoelectric device providing a thermal grill illusion to the user, and is a case in which a ratio between the number of thermoelectric elements included in the thermoelectric group which outputs cold heat to the user and the number of thermoelectric elements included in the thermoelectric group which outputs warm heat to the user is 1:1.5. Data expressed as quadrangles is a temperature of the heat dissipation module of the thermoelectric device providing a feeling of coldness to the user, and since there are only the thermoelectric groups which output cold heat, it can be seen that a ratio between the number of thermoelectric elements included in the thermoelectric group which outputs cold heat to the user and the number of thermoelectric elements included in the thermoelectric group which outputs warm heat to the user is 1:0. An x-axis of the graph refers to a time in which a time point at which the thermoelectric device starts to provide a thermal stimulus is regarded as 0, and a y-axis refers to a temperature measured in one region of the heat dissipation sheet. Further, the thermoelectric device used to measure the data in FIG. 14 includes a thin plate-shaped heat dissipation module (hereinafter, referred to as a ‘heat dissipation sheet’) in which a fin structure is not formed like the thermoelectric device shown in FIG. 12.

The thermoelectric device providing a thermal grill illusion may provide heat dissipation performance sufficient for performing heat dissipation even while dissipating heat using the heat dissipation sheet compared to the thermoelectric device providing a feeling of coldness. Referring to FIG. 14, it can be seen that a temperature of the heat dissipation sheet of the thermoelectric device providing the feeling of coldness sharply rises, for example, exceeds 40° C. within 20 seconds, but a temperature of the heat dissipation sheet of the thermoelectric device providing the thermal grill illusion exceeds 30° C. after 40 seconds or more. Further, it can be seen that the temperature of the heat dissipation sheet of the thermoelectric device providing the feeling of coldness rises to 60° C. or more after 100 seconds and to 70° C. or more after 200 seconds, but the temperature of the heat dissipation sheet of the thermoelectric device providing the thermal grill illusion rises only to 40° C. or less after 100 seconds and to 45° C. or less after 200 seconds.

A ratio between the number of N-type semiconductors and P-type semiconductors included in the first thermoelectric group and the number of N-type semiconductors and P-type semiconductors included in the second thermoelectric group may be 0.2 to 4. Alternatively, a ratio between the number of N-type semiconductors and P-type semiconductors included in the first thermoelectric group and the number of N-type semiconductors and P-type semiconductors included in the second thermoelectric group may be 0.5 to 3. Alternatively, a ratio between the number of N-type semiconductors and P-type semiconductors included in the first thermoelectric group and the number of N-type semiconductors and P-type semiconductors included in the second thermoelectric group may be 1 to 2. Alternatively, a ratio between the number of N-type semiconductors and P-type semiconductors included in the first thermoelectric group and the number of N-type semiconductors and P-type semiconductors included in the second thermoelectric group may be 1.3 to 1.7.

A thermoelectric group including a greater number of N-type semiconductors and P-type semiconductors among the first thermoelectric group and the second thermoelectric group may provide a feeling of warmth toward the first surface. In this case, the heat dissipation performance of the thermoelectric module may be improved compared to the opposite case. Alternatively, the user may feel a more vivid thermal grill illusion.

The temperature of the thermoelectric device (for example, the temperature of the heat dissipation module) may vary according to the areas of the thermal stimulus providing regions. For example, the temperature of the heat dissipation module included in the thermoelectric device may vary by adjusting the areas of the thermal stimulus providing region of the thermoelectric group providing warm heat and the thermal stimulus providing region of the thermoelectric group providing cold heat.

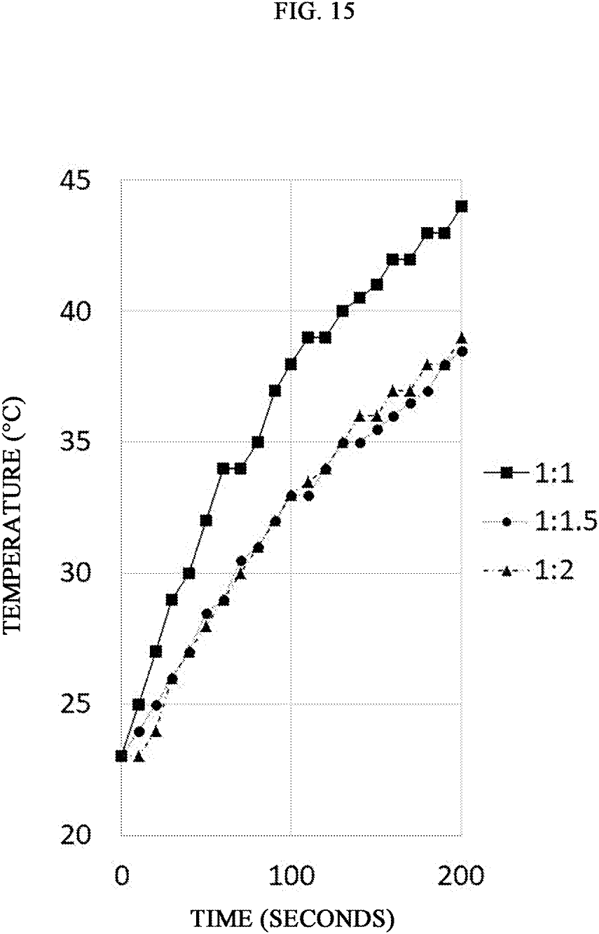

FIG. 15 is a graph illustrating a temperature change of the heat dissipation module included in the thermoelectric device providing the thermal grill illusion according to one embodiment. In FIG. 15, a size of the thermal stimulus providing region may vary according to the type of thermal stimulus provided by the thermoelectric group, and the thermoelectric groups providing the same thermal stimulus are manufactured to have the thermal stimulus providing regions of the same area. A ratio between the area of the thermal stimulus providing region of the thermoelectric group providing a feeling of coldness to the user and the area of the thermal stimulus providing region of the thermoelectric group providing a feeling of warmth to the user is expressed as a:b. For example, a case in which the ratio is 1:1.5 means that the area of the thermal stimulus providing region of the thermoelectric group providing the feeling of warmth is 1.5 times larger compared to the area of the thermal stimulus providing region of the thermoelectric group providing the feeling of coldness to the user. Further, an x-axis of the graph refers to a time in which a time point at which the thermoelectric device starts to provide a thermal stimulus is regarded as 0, and a y-axis refers to a temperature measured in one region of the heat dissipation sheet. The thermoelectric device used to measure the data in FIG. 15 includes a heat dissipation sheet similar to that of the thermoelectric device shown in FIG. 12.

Referring to FIG. 15, when the area of the thermal stimulus providing region of the thermoelectric group providing the feeling of warmth is larger than the area of the thermal stimulus providing region of the thermoelectric group providing the feeling of coldness, a temperature increase of the heat dissipation sheet may decrease. A temperature of the thermoelectric device with a ratio of 1:1 exceeds 30° C. after only 40 seconds, but temperatures of the thermoelectric devices with ratios of 1:1.5 and 1:2 exceed 30° C. after 70 seconds. Further, it can be seen that the temperature of the thermoelectric device with a ratio of 1:1 rises to approximately 38° C. after 100 seconds and approximately 45° C. after 200 seconds, but the temperatures of the thermoelectric devices with ratios of 1:1.5 and 1:2 rise only to approximately 33° C. after 100 seconds and approximately 38° C. after 200 seconds

In FIG. 15, although only the cases in which the ratios between the area of the thermal stimulus providing region of the thermoelectric group providing the feeling of coldness to the user and the area of the thermal stimulus providing region of the thermoelectric group providing the feeling of warmth to the user are 1:1, 1:1.5, and 1:2 are shown, the ratios are not limited thereto. For example, the ratio may be 1:2 or more, for example, 1:3, 1:4, 1:5, 1:10, 1:50, 1:100, or more. Alternatively, the ratio may be 1:1 or less, for example, 1:0.5, 1:0.25, 1:0.1, 1:0.05, 1:0.01, or less.

The area of the thermal stimulus providing region may depend on the number of thermoelectric elements included in the thermoelectric group. For example, when a thermoelectric device is manufactured using a thermoelectric group including thermoelectric elements of the same size, a case in which a ratio between an area of the thermal stimulus providing region of the first thermoelectric group providing a feeling of coldness to the user and an area of the thermal stimulus providing region of the second thermoelectric group providing a feeling of warmth to the user is a:b may mean that a ratio between the number of thermoelectric elements included in the first thermoelectric group and the number of thermoelectric elements included in the second thermoelectric group is a:b. The ratio between the number of thermoelectric elements included in the thermoelectric group providing the feeling of coldness to the user and the number of thermoelectric elements included in the thermoelectric group providing the feeling of warmth may be 1:1 or more, for example, 1:2, 1:3, 1: 4, 1:5, 1:10, 1:50, 1:100 or more. Alternatively, the ratio may be 1:1 or less, for example 1:0.5, 1:0.25, 1:0.1, 1:0.05, 1:0.01 or less.

Although FIGS. 10 and 11 and the like described above illustrate that the sizes of the thermoelectric elements are the same, the sizes of the thermoelectric elements may be different.

The sizes of the thermoelectric elements may vary according to the thermoelectric group. The sizes of the thermoelectric elements included in the thermoelectric group may vary according to the type of the thermal stimulus provided to the user by the thermoelectric group, for example, the sizes of the thermoelectric elements included in the thermoelectric group providing the feeling of warmth to the user may be larger than the sizes of the thermoelectric elements included in the thermoelectric group providing the feeling of coldness or vice versa. Here, a size change of the thermoelectric element may include at least one of a change in an area of a bottom surface and a change in a height in the case of the pillar-shaped thermoelectric element.

Even in the thermal stimulus providing regions having the same area, the thermal stimulus may vary as the size of the thermoelectric element is changed. For example, when the sizes of the thermal stimulus providing regions are the same, the intensity of the thermal stimulus may increase as the sizes of the thermoelectric elements increase.

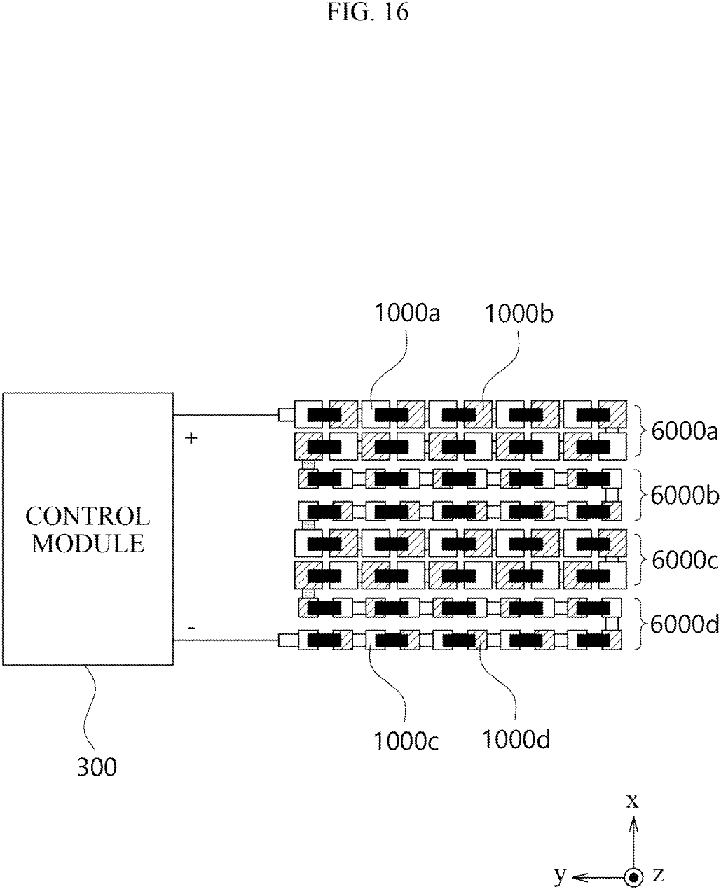

FIG. 16 is a plan view related to a thermoelectric device including thermoelectric elements having different sizes according to one embodiment. Referring to FIG. 16, widths of the bottom surfaces of the thermoelectric elements 1000a and 1000b included in the first thermoelectric group 6000a and the third thermoelectric group 6000c may be larger than widths of the bottom surfaces of the thermoelectric elements 1000c and 1000d included in the second thermoelectric group 6000b and the fourth thermoelectric group 6000d. In consideration of a connection relationship between the thermoelectric elements of FIG. 16, the first thermoelectric group 6000a and the third thermoelectric group 6000c may provide any one of a feeling of warmth and a feeling of coldness, and the second thermoelectric group 6000b and the fourth thermoelectric group 6000d may provide the other of the feeling of warmth and the feeling of coldness.

A temperature change of the thermoelectric device may vary by adjusting the size of the thermoelectric device. For example, a temperature increase of the thermoelectric device may be decreased by adjusting the size of the thermoelectric element.

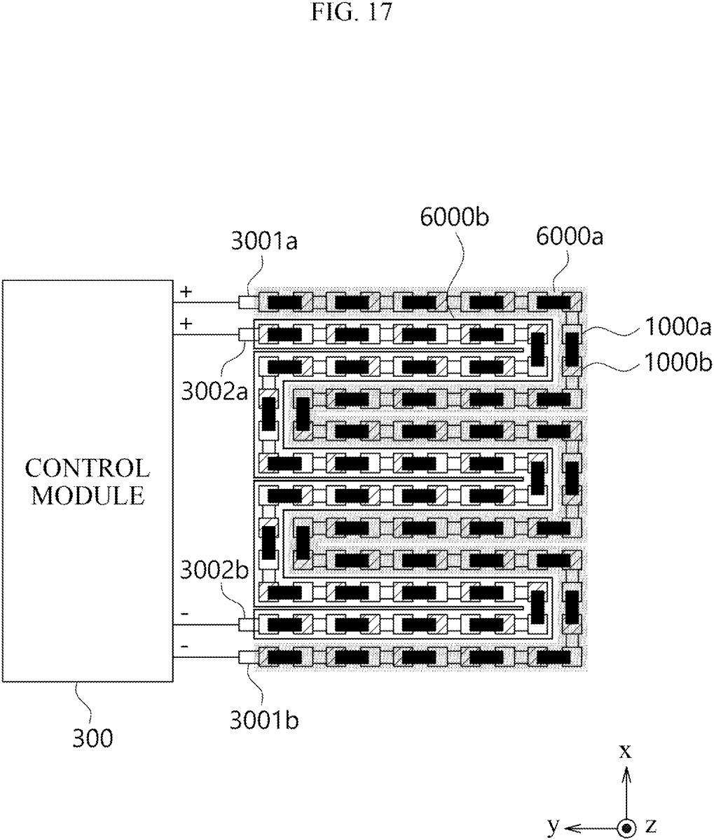

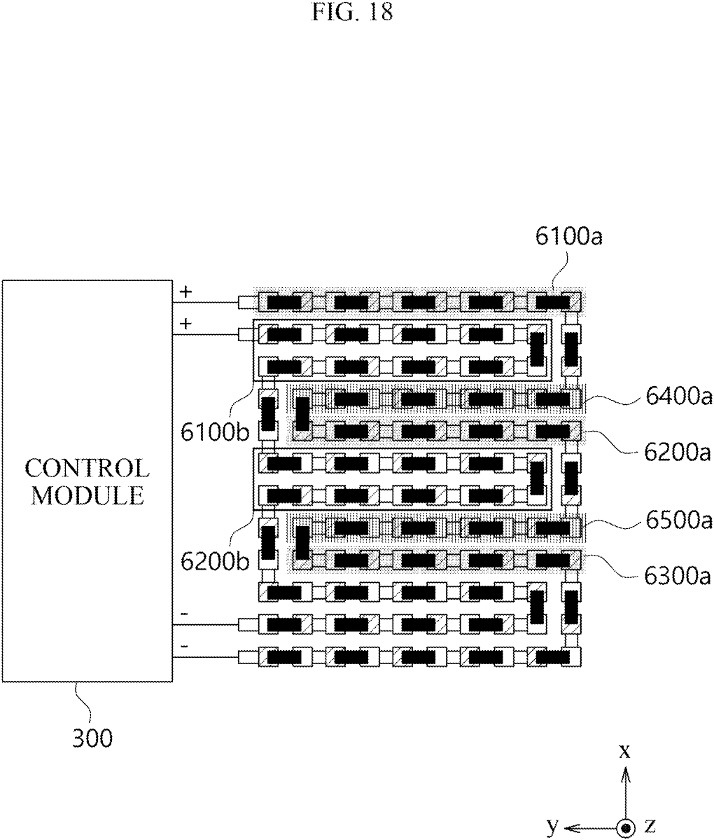

FIGS. 17 and 18 are views for describing a thermoelectric element disposition pattern of a thermoelectric module including two pairs of terminals according to one embodiment.

The thermoelectric module may include two thermoelectric groups each connected to a pair of terminals. Referring to FIG. 17, the first thermoelectric group 6000a may be connected to a first terminal 3001a and a second terminal 3001b, and the second thermoelectric group 6000b may be connected to a third terminal 3002a and a fourth terminal 3002b.

The thermoelectric group may include thermoelectric elements disposed in a first direction and thermoelectric elements disposed in a second direction perpendicular to the first direction. Here, the direction in which the thermoelectric elements are disposed refers to a direction in which the thermoelectric elements are electrically connected through the electrode, for example, a case in which the thermoelectric elements are disposed in the first direction means that the thermoelectric element-the electrode-the thermoelectric element are electrically connected and disposed along the first direction. Referring to FIGS. 17 and 18 , the first thermoelectric group 6000a may include a thermoelectric element disposed to proceed in a (-y) direction from the first terminal 3001a, a thermoelectric element disposed to proceed in a (-x) direction perpendicular to the (-y) direction, a thermoelectric element disposed to proceed in a (+y) direction perpendicular to the (-x) direction, and a thermoelectric element disposed to proceed in a (-x) direction perpendicular to the (+y) direction, and may be connected to the second terminal 3001b after this pattern repeatedly proceeds. Thermoelectric elements included in the second thermoelectric group 6000b may also be disposed like the above.

Between the thermoelectric elements disposed to proceed in a specific direction in a specific thermoelectric group, thermoelectric elements of another thermoelectric group may be disposed. Referring to FIGS. 17 and 18, the first thermoelectric group 6000a may include first sub-thermoelectric groups 6100a, 6200a, and 6300a including a plurality of thermoelectric elements disposed in the (-y) direction, and the second thermoelectric group 6000b may include second sub-thermoelectric groups 6100b and 6200b disposed between the first sub-thermoelectric groups 6100a, 6200a, and 6300a.

Between the thermoelectric elements disposed to proceed in the specific direction in the specific thermoelectric group, thermoelectric elements included in the same thermoelectric group but disposed to proceed in a different direction may be disposed. Referring to FIGS. 17 and 18, the first thermoelectric group 6000a may include third sub-thermoelectric groups 6400a and 6500a including a plurality of thermoelectric elements disposed between the first sub-thermoelectric groups 6100a, 6200a, and 6300a but disposed to proceed in the (+y) direction different from the first sub-thermoelectric groups 6100a, 6200a, and 6300a.

The thermoelectric module including the thermoelectric elements and two pairs of terminals disposed like the above may provide a thermal grill illusion to the user. The thermal grill illusion may be provided as the thermoelectric module includes the first thermoelectric group and the second thermoelectric group which alternately appear in one direction. Referring to FIG. 17, it can be seen that the first thermoelectric group 6000a and the second thermoelectric group 6000b are alternately disposed along an x-axis direction. Here, since the first thermoelectric group 6000a provides any one of warm heat and cold heat to the user, and the second thermoelectric group 6000b provides the other of the warm heat and the cold heat to the user, the thermoelectric module may provide the thermal grill illusion to the user.

The thermoelectric module may provide a feeling of warmth and a feeling of coldness in addition to the thermal grill illusion to the user by adjusting polarities of the power applied to the terminals. For example, when the polarities of the power applied to the first terminal 3001a and the second terminal 3001b are maintained as shown in FIG. 17, but the polarities of the power applied to the third terminal 3002a and the fourth terminal 3002b are changed to be opposite FIG. 17, since both the first thermoelectric group 6000a and the second thermoelectric group 6000b provide warm heat or cold heat to the user, the thermoelectric module may provide the feeling of warmth or the feeling of coldness to the user.

In the above, the configuration and features of the present invention have been described with respect to the embodiments, but the present invention is not limited thereto, and it is apparent that those skilled in the art may perform various changes or modifications within the spirit and scope of the present invention, and accordingly, it is noted that these changes or modifications belong to the accompanying claims.

1. A thermoelectric device including a plurality of thermoelectric groups connected in series between a first terminal and a second terminal, the first terminal and the second terminal provided in a plate shape deformable to a curved surface, having a first surface and a second surface opposite the first surface as a main surface, and configured to receive power from the outside, including:
wherein the plurality of thermoelectric groups include:
a first thermoelectric group configured to provide any one of a feeling of coldness and a feeling of warmth toward the first surface when the power is applied, and including an N-type semiconductor and a P-type semiconductor disposed between the first surface and the second surface, and an electrode configured to alternately connect an N-type semiconductor and a P-type semiconductor and disposed adjacent to any one surface of the main surface, and
a second thermoelectric group providing the other of the feeling of coldness and the feeling of warmth toward the first surface when the power is applied, and including an N-type semiconductor and a P-type semiconductor disposed between the first surface and the second surface, and an electrode configured to alternately connect an N-type semiconductor and a P-type semiconductor and disposed adjacent to any one surface of the main surfaces,
wherein one end electrically adjacent to the first terminal of an electrode disposed adjacent to the first surface among the electrode of the second thermoelectric group is connected to the P-type semiconductor, when one end electrically adjacent to the first terminal of an electrode disposed adjacent to the first surface among the electrode of the first thermoelectric group is connected to the N-type semiconductor,
wherein the one end electrically adjacent to the first terminal of the electrode disposed adjacent to the first surface among the electrode of the second thermoelectric group is connected to the N-type semiconductor, when the one end electrically adjacent to the first terminal of the electrode disposed adjacent to the first surface among the electrode of the first thermoelectric group is connected to the P-type semiconductor, and
wherein the first thermoelectric group and the second thermoelectric group are connected through a connection electrode, and the connection electrode connects the N-type semiconductor of the first thermoelectric group and the N-type semiconductor of the second thermoelectric group or connects the P-type semiconductor of the first thermoelectric group and the P-type semiconductor of the second thermoelectric group.

2. The thermoelectric device of claim 1, wherein a number of N-type semiconductors and P-type semiconductors included in the first thermoelectric group is greater than a number of N-type semiconductors and P-type semiconductors included in the second thermoelectric group.

3. The thermoelectric device of claim 1, wherein a ratio between a number of N-type semiconductors and P-type semiconductors included in the first thermoelectric group and a the number of N-type semiconductors and P-type semiconductors included in the second thermoelectric group is 0.2 to 4.

4. The thermoelectric device of claim 3, wherein a thermoelectric group including a greater number of N-type semiconductors and P-type semiconductors among the first thermoelectric group and the second thermoelectric group is configured to provide a feeling of warmth toward the first surface.

5. The thermoelectric device of claim 1,
wherein the first surface includes a plurality of thermal stimulus providing regions respectively corresponding to the plurality of thermoelectric groups, and
wherein an area of the thermal stimulus providing region corresponding to the first thermoelectric group is larger than an area of the thermal stimulus providing region corresponding to the second thermoelectric group.

6. The thermoelectric device of claim 1, wherein a ratio between an area of the thermal stimulus providing region corresponding to the first thermoelectric group and an area of the thermal stimulus providing region corresponding to the second thermoelectric group may be 0.2 to 4.

7. The thermoelectric device of claim 6, wherein a thermoelectric group having a larger area of the corresponding thermal stimulus providing region among the first thermoelectric group and the second thermoelectric group is configured to provide a feeling of warmth toward the first surface.

8. The thermoelectric device of claim 1, wherein the thermoelectric device is configured to provide a thermal grill illusion to a user through the first surface.

9. The thermoelectric device of claim 1, wherein the thermoelectric device further includes a heat dissipation sheet disposed on the second surface.

10. The thermoelectric device of claim 9, wherein a heat dissipated toward the second surface by the thermoelectric group providing the feeling of coldness toward the first surface among the plurality of thermoelectric groups is transferred to the thermoelectric group providing the feeling of warmth toward the first surface among the plurality of thermoelectric groups through the heat dissipation sheet.

11. The thermoelectric device of claim 9, wherein, the heat dissipation sheet is a single sheet, and the plurality of thermoelectric groups is disposed on one surface of the single sheet.

12. The thermoelectric device of claim 11, wherein the other surface of the single sheet is formed as a flat surface and have a thickness of 0.1 to 20 millimeters.
